(19) United States
(12) Patent Application Publication
(10) Pub. No.: US 2013/0045514 A1
(43) Pub. Date: Feb. 21, 2013

(54) Biologically Catalyzed Mineralization of Carbon Dioxide

(71) Applicants: Roberto Barbero, Cambridge, MA (US); Elizabeth Wood, Cambridge, MA (US); Angela Belcher, Lexington, MA (US)

(72) Inventors: Roberto Barbero, Cambridge, MA (US); Elizabeth Wood, Cambridge, MA (US); Angela Belcher, Lexington, MA (US)

(21) Appl. No.: 13/211,910

(22) Filed: Aug. 17, 2011

(51) Int. Cl. C12P 9/00; C12M 1/00

(52) U.S. Cl. 435/131; 435/289.1

(57) ABSTRACT

Carbonic anhydrase can be expressed on a cell surface in a system and method for mineralizing carbon dioxide. The system and method can optionally include a mineralization peptide to facilitate formation of minerals from carbonate ions and divalent metal cations.

### TECHNICAL FIELD

The present invention relates to biologically catalyzed mineralization of carbon dioxide.

### BACKGROUND

Since the middle of the nineteenth century, the concentration of atmospheric carbon dioxide (CO₂) has increased from 280 parts per million (ppm) to 380 ppm. CO₂ is a greenhouse gas and it is widely accepted that rising atmospheric CO₂ levels are responsible for increasing average global temperatures. Climate scientists believe that if atmospheric CO₂ levels and global temperatures continue to rise, there will be serious and irrevocable damage to the Earth's ecosystems. Reducing emissions of CO₂ into the atmosphere can help mitigate these problems.

Burning of fossil fuels is one of the largest overall contributors to CO₂ emissions, and fossil-fuel fired power plants are the largest energy-related emitters of CO₂. Thus, preventing the CO₂ generated by such power plants from being emitted into the atmosphere is critical in the battle against global warming.

Several technologies for transporting and storing large volumes of CO₂ have progressed beyond the research stage. Additionally, several CO₂ capture technologies are already mature enough to be considered economically viable in certain situations. For example, transporting large volumes of liquid or gaseous CO₂ from a capture point to a storage point via a pipeline could be achieved using the same technologies that the oil industry already uses to move oil and natural gas. As part of a process called enhanced oil recovery (EOR), the CO₂ can then be pumped into an underground oil bed to help extract additional oil while simultaneously storing the CO₂ in a geological reservoir, sequestered from the atmosphere.

The two most promising locations for long-term CO₂ storage are in deep underground geological formations, or in the ocean. Both of these strategies carry legitimate risks of CO₂ leakage back into the atmosphere; and these sites will require long-term monitoring.

Storage capacity and time are important considerations for CO₂ storage technologies. At current emission rates, EOR is capable of storing no more several years' worth of CO₂ emissions. Mineral carbonation has a significant storage capacity (theoretically enough to store all CO₂ emissions of the twenty-first century) and long storage time (on the order of thousands of years).

Mineral carbonation entails the conversion of CO₂ to solid carbonate minerals, generally a four-step process:

CO_{2(g)} [symbol] CO₂(aq) (1)

CO_{2(aq)}+H₂O [symbol] HCO₃⁻+H⁺ (2)

HCO₃⁻ [symbol] CO₃²⁻+H⁺ (3)

CO₃²⁻+M²⁺→MCO₃ (4)

where M is a metal such as Mg or Ca. Mineral carbonation has not a feasible option for industrial CO₂ sequestration because without catalysis, the mineralization process occurs slowly, or requires extreme and costly operating conditions.

### SUMMARY

In one aspect, a system for the mineralization of carbon dioxide includes a reactor containing an aqueous cell composition including a cell expressing a carbonic anhydrase on the cell surface; a carbon dioxide source configured to supply carbon dioxide to the reactor; and an aqueous metal ion composition including divalent metal cations, where the aqueous cell composition and the aqueous metal ion composition are optionally part of the same aqueous composition.

In another aspect, a method of mineralizing carbon dioxide includes providing an aqueous cell composition including a cell expressing a carbonic anhydrase on the cell surface, contacting the aqueous cell composition with carbon dioxide, thereby producing aqueous carbonate ions, and contacting the aqueous carbonate ions with divalent metal cations.

The cell expressing the carbonic anhydrase can be a yeast cell. The aqueous metal ion composition can further include a mineralization peptide. The mineralization peptide can be expressed on a cell surface. The mineralization peptide can be expressed on the surface of the cell expressing the carbonic anhydrase; or on the surface of a different cell.

The system can further include a separator configured to separate the cell from a solute in the aqueous composition including the cell, and a second reactor containing the aqueous metal ion composition (for example, when the aqueous cell composition and the aqueous metal ion composition are not part of the same aqueous composition). The carbon dioxide source can include a flue gas.

The method can further include contacting the aqueous carbonate ions and the divalent metal cations with a mineralization peptide. In the method, contacting the aqueous cell composition with carbon dioxide can include contacting the aqueous cell composition with a flue gas. The method can further include separating the cell expressing a carbonic anhydrase on the cell surface from the aqueous carbonate ions prior to contacting the aqueous carbonate ions with the divalent metal cations. The separated cell can be returned to the aqueous cell composition.

Other aspects, embodiments, and features will become apparent from the following description, the drawings, and the claims.

### BRIEF DESCRIPTION OF THE DRAWINGS

FIGS. 1A and 1B are schematic depictions of systems for mineralization of CO₂.

FIG. 2 is a graph showing activity of carbonic anhydrase II expressed on the surface of S. cerevisiae.

FIGS. 3A-3B are microscopic images of calcium carbonate formed in the presence and absence of yeast cells, respectively.

FIGS. 4A-4D are microscopic images of calcium carbonate formed in the presence of yeast cells.

### DETAILED DESCRIPTION

In general, mineralization of CO₂ can be facilitated by biological catalysis. Reactions (2) and (4) above are biologically catalyzed by some organisms. Reaction (2), hydration of dissolved CO₂ to produce bicarbonate and H⁺, is catalyzed by the enzyme carbonic anhydrase. Reaction (4) is catalyzed by mineralization peptides found in, for example, mollusks, sea urchins, corals, and oysters. Like most biological catalysts, these operate efficiently in aqueous solutions at standard temperature and pressure. When used together, these can provide a system in which both hydration of aqueous CO₂, and formation of carbonate minerals, occur at a faster rate than they would in the absence of a catalyst.

Others have considered using whole organisms to biomineralize CO₂ for sequestration. For example, bacteria and cyanobacteria suspected of being capable of biomineralization have been screened for the ability to remove CO₂ from a closed reactor. B. D. Lee, et al., Biotechnology Progress, 20(5):1345-1351, 2004; T. J. Phelps, et al., Technical report, Oak Ridge National Laboratory, 2003; and Y. Roh, et al., Technical report, National Energy Technology Laboratory, 2000, each of which is incorporated by reference in its entirety. The species that were identified took several days to have a detectable impact on the CO₂ levels in a small reactor.

The use of enzymes for CO₂ capture has met with limited success (see, e.g., R. M. Cowan, et al., Ann NY Acad Sci, 984(1):453-469, 2003; and E. Kintisch, Science, 317(5835):186-186, 2007; each of which is incorporated by reference in its entirety). See also U.S. Pat. Nos. 7,803,575; 7,132,090; and 7,919,064; US Patent Application Publication Nos. 2010/0297723; 2011/0104779; 2010/0047866; 2010/0209997; and 2010/0120104; and C. Prabhu, et al., Energy Fuels, 25(3):1337-1342 (2011); F. A. Simsek-Ege, et al., J. Biomater. Sci., Polym. Ed., 13(11): 1175-1187 (2002); and G. M. King, Trends Microbiol., 19(2): 75-84 (2011); each of which is incorporated by reference in its entirety.

A system for mineralization of CO₂ can include a carbonic anhydrase for converting CO₂ to aqueous bicarbonate (HCO₃⁻). In aqueous environments, an equilibrium exists between bicarbonate and carbonate (CO₃²⁻). The carbonate formed can be subsequently mineralized with divalent metal cations (e.g., M²⁺) and optionally in the presence of a mineralization peptide. Thus, a system can include a CO₂ source, an aqueous composition including a carbonic anhydrase, and an aqueous composition including divalent metal cations and optionally including a mineralization peptide. As discussed below, the carbonic anhydrase can be in the same or in a separate aqueous composition as the divalent metal cations.

The CO₂ source can be a CO₂-containing gas (e.g., flue gases from a fossil fuel power plant) or CO₂ dissolved in a solvent (including, for example, an aqueous solvent). The CO₂-containing gas can be directly contacted with the aqueous composition including a carbonic anhydrase; or, in some cases, the CO₂-containing gas can be first contacted with an aqueous composition to afford a composition including aqueous CO₂. The composition including aqueous CO₂ can be subsequently contacted or combined with the aqueous composition including a carbonic anhydrase.

The aqueous composition can further include divalent metal cations (e.g., M²⁺), leading to formation of a carbonate mineral (MCO₃). This process can be facilitated by a mineralization peptide.

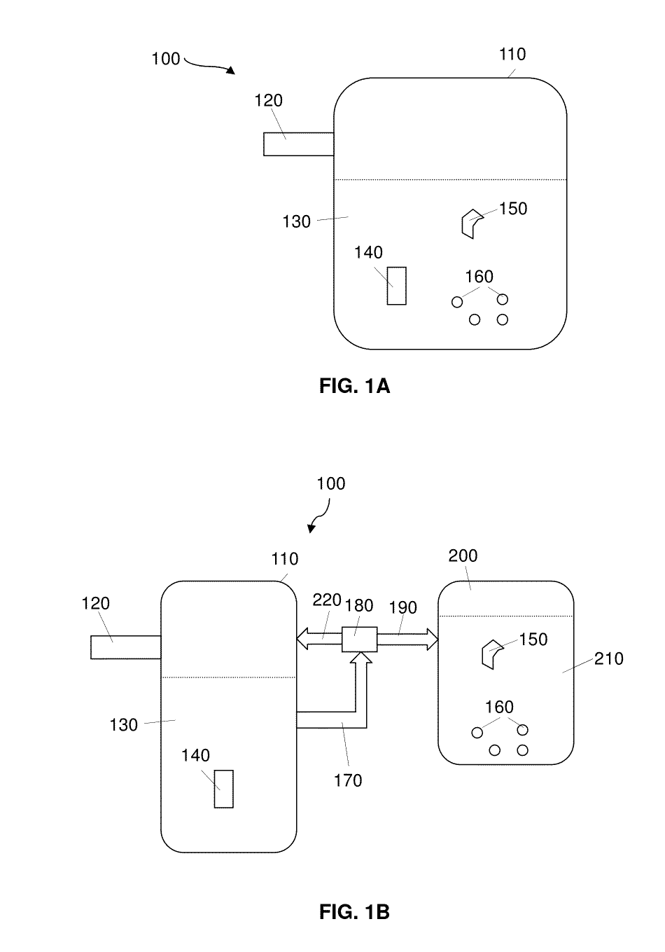

FIG. 1A illustrates system 100 for mineralization of CO₂. The system includes reactor 110 connected to CO₂ source 120. Reactor 110 also includes aqueous composition 130. Aqueous composition 130 includes carbonic anhydrase 140, mineralization peptide 150, and divalent metal cations 160. During operation, CO₂ from CO₂ source 120 comes into contact with aqueous composition 130 within reactor 110, and becomes dissolved in the aqueous composition. Once dissolved, carbonic anhydrase 140 catalyzes the conversion of CO₂ to HCO₃⁻, which is in equilibrium with CO₃²⁻. Combination of CO₃²⁻ with divalent metal cations 160 produces a carbonate mineral; this combination is facilitated by optional mineralization peptide 150.

FIG. 1B illustrates an alternate configuration of system 100, which includes reactor 110 and reactor 200. In this configuration, reactor 110 is connected to CO₂ source 120, and includes aqueous composition 130. Aqueous composition 130 includes carbonic anhydrase 140. Reactor 110 is also connected to withdrawal channel 170, which is connected in turn to separator 180. Separator 180 is further connected to return channel 220, which is connected to reactor 110. Separator 180 is also connected to delivery channel 190, which is connected to reactor 200. Reactor 200 includes aqueous composition 210. Aqueous composition 210 includes divalent metal cations 160 and optional mineralization peptide 150.

During operation using this configuration, CO₂ from CO₂ source 120 comes into contact with aqueous composition 130 within reactor 110, and becomes dissolved in the aqueous composition. Once dissolved, carbonic anhydrase 140 catalyzes the conversion of CO₂ to HCO₃, which is in equilibrium with CO₃²⁻. A portion of aqueous composition 130 is diverted to withdrawal channel 170 and delivered to separator 180. In separator 180, carbonic anhydrase is separated from HCO₃⁻. The separation is such that a portion of the aqueous composition which is relatively enriched with carbonic anhydrase 140, but relatively diminished with HCO₃⁻, is returned to reactor 110 via return channel 220. The portion returned combines with aqueous composition 130. The returned carbonic anhydrase 140 retains catalytic activity.

A different portion of the aqueous composition, which is relatively enriched with HCO₃⁻, but relatively diminished with carbonic anhydrase, is delivered to reactor 200 via delivery channel 190. Within reactor 200, combination of CO₃²⁻ with divalent metal cations 160 produces a carbonate mineral; this combination is facilitated by mineralization peptide 150.

Reactors 110 and 200 can independently be, for example, a tray column reactor, a packed column reactor, a spray column reactor, or a bubble column reactor. The system can be, for example, a batch or continuous reactor system. A continuous system can be preferred, such as when removing CO₂ from an exhaust stream. System 100 can further include components for monitoring conditions within the system, e.g., temperature, flow rates, concentration of various compounds (such as CO₂ or divalent metal cations), or concentration of the host organism; and components for delivering or removing additional materials, e.g., a source for delivering nutrients to the host organism.

Numerous carbonic anhydrases are known, including different isoforms from the same organism. Any of these can be used, as can variants, e.g., mutants, fusion proteins, chemically modified forms, provided the necessary catalytic activity is present.

The carbonic anhydrase can be heterologously expressed in a non-native organism. In other words, the carbonic anhydrase can be produced by genetic engineering of a host organism. The host organism can be a microorganism, e.g., a unicellular microorganism such as bacteria, cyanobacteria, a unicellular fungus, or the like. The unicellular microorganism can be a free-living organism, i.e., one that can survive, grow, and/or reproduce without the need to be anchored to a surface. Suitable a unicellular fungi can include yeasts, such as Saccharomyces cerevisiae.

The carbonic anhydrase can be used in isolated form (e.g., where the protein has been purified prior to use), in a crude mixture (e.g., cell lysate), or in a biological medium, e.g., where cells expressing the carbonic anhydrase are present in the system for mineralization of CO₂. The host organism can be engineered such that the carbonic anhydrase is retained within the cell, excreted from the cell (e.g., by exocytosis, transport, a transmembrane translation process, or by cell rupture), or expressed on the cell surface (i.e., exposed to the extracellular medium while anchored to a cell membrane or cell wall). For example, S. cerevisiae can be engineered so as to express a desired polypeptide on the cell wall (see, for example, E. T. Boder and K. D. Wittrup., Nature Biotechnology, 15:553-557, 1997; E. T. Boder and K. D. Wittrup, Applications of Chimeric Genes and Hybrid Proteins, Pt C, 328:430-444, 2000; and G. Chao, et al., Nature Protocols, 1(2):755-768, 2006; each of which is incorporated by reference in its entirety. Proteins with sizes similar to carbonic anhydrase II can be expressed on the surface of S. cerevisiae at levels of at least 10,000-50,000 proteins per cell (see, for example, R. Parthasarathy, et al., Biotechnology Progress, 21(6):1627-1631, 2005, which is incorporated by reference in its entirety).

Accordingly, aqueous composition 130 can optionally be a growth medium selected to support survival, growth, and reproduction of the host organism, and expression of the carbonic anhydrase by the host organism.

In the configuration illustrated in FIG. 1B, carbonic anhydrase 140 can be conveniently separated from HCO₃₋ on the basis of size. In particular, when the carbonic anhydrase is expressed on the cell surface of a unicellular host organism, separator 180 can operate, e.g., by filtration, sedimentation, or other principle for separation of cell-sized particles from aqueous solutes such as HCO₃⁻.

A number of mineralization peptides that promote the formation of carbonate minerals are known, including crustocalcin (Penaeus japonicus), ansocalcin (anser anser), perlucin (Haliotis discus), and nacrein (Pinctada fucata). Any of these can be used, as can variants, e.g., mutants, fusion proteins, chemically modified forms, provided the necessary activity is present.

The mineralization peptide can be heterologously expressed in a non-native organism In other words, the mineralization peptide can be produced by genetic engineering of a host organism. The host organism can be a microorganism, e.g., a unicellular microorganism such as bacteria, cyanobacteria, a unicellular fungus, or the like. The unicellular microorganism can be a free-living organism, i.e., one that can survive, grow, and/or reproduce without the need to be anchored to a surface. Suitable a unicellular fungi can include yeasts, such as Saccharomyces cerevisiae.

The mineralization peptide can be used in isolated form (e.g., where the protein has been purified prior to use), in a crude mixture (e.g., cell lysate), or in a biological medium, e.g., where cells expressing the mineralization peptide are present in the system for mineralization of CO₂. The host organism can be engineered such that the mineralization peptide is retained within the cell, excreted from the cell (e.g., by exocytosis, transport, a transmembrane translation process, or by cell rupture), or expressed on the cell surface (i.e., exposed to the extracellular medium while anchored to a cell membrane or cell wall). As discussed above, S. cerevisiae can be engineered so as to express a desired polypeptide on the cell wall.

Carbonate minerals formed in the presence of yeast cells can exhibit different morphology than those formed in the absence of yeast, even when the yeast do not express a mineralization peptide. Advantageously, carbonate minerals formed in the presence of yeast cells can aggregate in larger particles, such that separation of the minerals from an aqueous composition (e.g., a suspension of mineral particles) is simplified. In some cases, the carbonate minerals can be attached to the yeast surface, even when the yeast do not express a mineralization peptide.

The mineralized tissues of many organisms often contain peptides rich in acidic amino acids and phosphorylated amino acids, though they occasionally also contain acidic sulfated polysaccharides or glycoproteins. See L. Addadi and S. Weiner. Angewandte Chemie Int. Ed. Engl., 31(2):153-169, 1992, which is incorporated by reference in its entirety. Mineralization on cell surfaces, mediated by cell-surface expressed mineralization peptides, is described in, e.g., E. M. Krauland, et al., Biotechnology and Bioengineering, 97(5):1009-1020, 2007; K. T. Nam, et al., ACS Nano, 2(7):1480-1486, 2008; B. R. Peelle, et al., Acta Biomaterialia, 1(2):145-154, 2005; B. R. Peelle, et al., Langmuir, 21(15):6929-6933, 2005; each of which is incorporated by reference in its entirety.

Mineralization peptides can be rich in aspartate and glutamate, and can appear in repeated motifs. For example, in the scallop shell protein MSP-1, the aspartate residues are arranged with repeats such as Asp-Gly-Ser-Asp and Asp-Ser-Asp. The regular arrangements of carboxylate groups can be important for the growth of calcium carbonate. See, e.g., I. Sarashina and K. Endo. Marine Biotechnology, 3(4):362-369, 2001, which is incorporated by reference in its entirety. In the protein nacrein, which assists in the mineralization of calcium carbonate in oysters, the repeated domain of Gly-Xaa-Asn (Xaa=Asp, Asn, or Glu) was identified, which has been proposed to bind calcium and participate in calcium carbonate formation (H. Miyamoto, et al., PNAS, 93(18):9657-9660, 1996, which is incorporated by reference in its entirety). These repeated domains can be relatively small, on the order of ten to twenty amino acids. Previous work with yeast-surface-displayed peptides demonstrated that peptides that are as small as twelve amino acids can interact with minerals (E. M. Krauland, et al., Biotechnology and Bioengineering, 97(5):1009-1020, 2007; K. T. Nam, et al., ACS Nano, 2(7):1480-1486, 2008, which is incorporated by reference in its entirety). Thus, small peptides utilizing these repeated domains, and/or simple repeats of glutamate and aspartate, can be used as mineralization peptides, particularly when expressed on a cell surface.

### EXAMPLES

The cDNA for bovine carbonic anhydrase 2 (bCA2) and human carbonic anhydrase 2 (hCA2) were cloned into the yeast surface display plasmid pCT-CON2 using standard molecular biology techniques. All cloning steps were performed in Escherechia coli. BCA2 cDNA in the pCMV-SPORT6 plasmid was ordered from Open Biosystems (clone ID: 7985245; Accession number: BC103260). HCA2 cDNA in the pDONR221 plasmid was ordered from the Dana Farber/Harvard Cancer Center DNA Resource Core (plasmid ID: HsCD00005312; Refseq ID: NM 000067). The pCTCON2 plasmid was a generous gift from the Wittrup lab. It should be noted that both CA2 genes contained internal BamHI restriction sites, which were removed using a Stratagene Quikchange Lightning Site Directed Mutagenesis Kit to make them compatible with the yeast display vector, pCTCON2. The genes were PCR amplified from the plasmids, and an upstream NheI restriction site and a downstream BamHI restriction site were added to make them compatible with the pCTCON2 plasmid. The yeast display vector pCTCON2 and the bCA2 and hCA2 PCR products were digested with the appropriate restriction enzymes, and the digestion products were ligated into the vector. Correct insertion of the genes of interest were confirmed by DNA sequencing reactions prior to transformation of the pCTCON2-hCA2 and pCTCON2-bCA2 plasmids into competent EBY100 S. cerevisiae cells. Transformed cells were propagated in SD-CAA media. Expression of the hCA2 and bCA2 enzymes was induced by transferring the cells to fresh SG-CAA media and growing them for 24 hours at 22° C.

Expression of genes from the pCTCON2 plasmid led to proteins that were fused to the N-terminal end of the Aga2 protein, a yeast mating protein that is permanently anchored to the surface of the yeast cell. In addition, the fusion protein had two epitope tags, an HA tag in between Aga2 and the gene of interest (carbonic anhydrase, in this case) and a c-MYC tag on the C-terminal end of the gene of interest. By staining the yeast cells with fluorescently labeled antibodies against these epitope tags, expression of the fusion protein and the protein of interest was confirmed. Fluorescent staining with an anti-HA antibody confirmed expression and display of the N-terminal end of the CA2 fusion proteins.

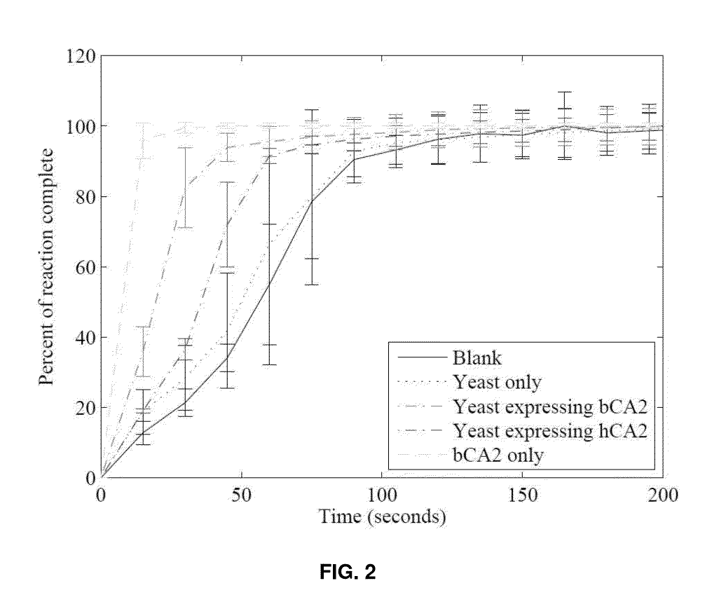

In order to test the activity of the carbonic anhydrase enzymes on the surface of the yeast cells, a modified version of the method developed by Wilbur and Anderson was used. See, e.g., K. M. Wilbur and N. G. Anderson., J. Biol. Chem., 176(1):147-154, 1948, which is incorporated by reference in its entirety. Briefly, the length of time required for CO₂-saturated water to lower the pH of a 0.012 M Tris-HCl buffered solution from 8.5 to 6.5 at 1° C. was monitored. The blank sample contained only the buffer and the CO₂-saturated water. All other samples had yeast or enzyme mixed into the buffer prior to the addition of the CO₂-saturated water. Each data point in FIG. 2 was the average of at least two runs. Error bars represent one standard deviation. In the absence of the enzyme, this reaction took about 2 minutes to reach 90% completion, whereas in the presence of purified bCA2 the reaction happened in less than 0.25 minutes (compare the dashed line with the solid black line in FIG. 2). The presence of the yeast cells expressing hCA2 or bCA2 also sped up the reaction, though to a lesser degree than purified bCA2 alone.

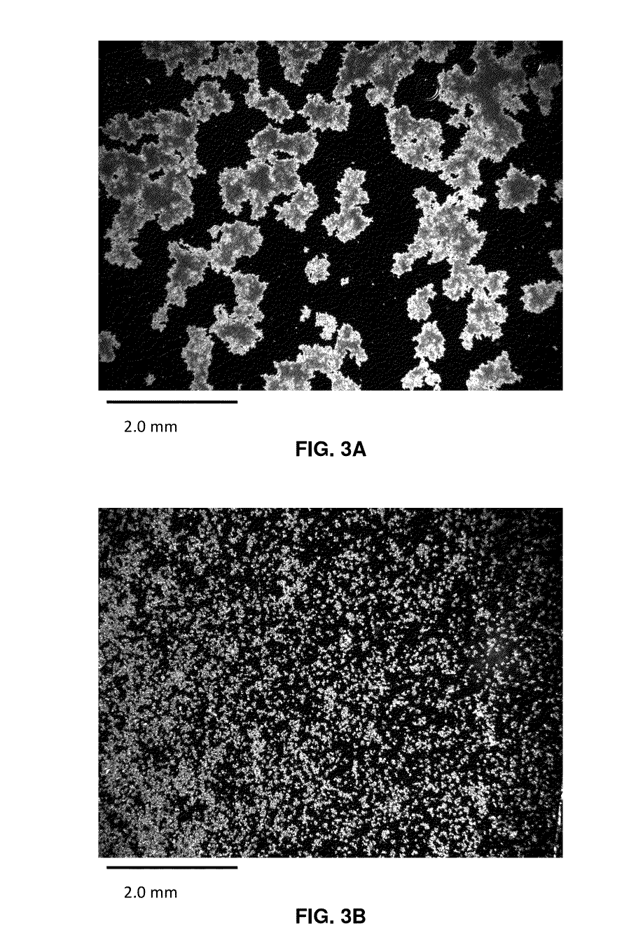

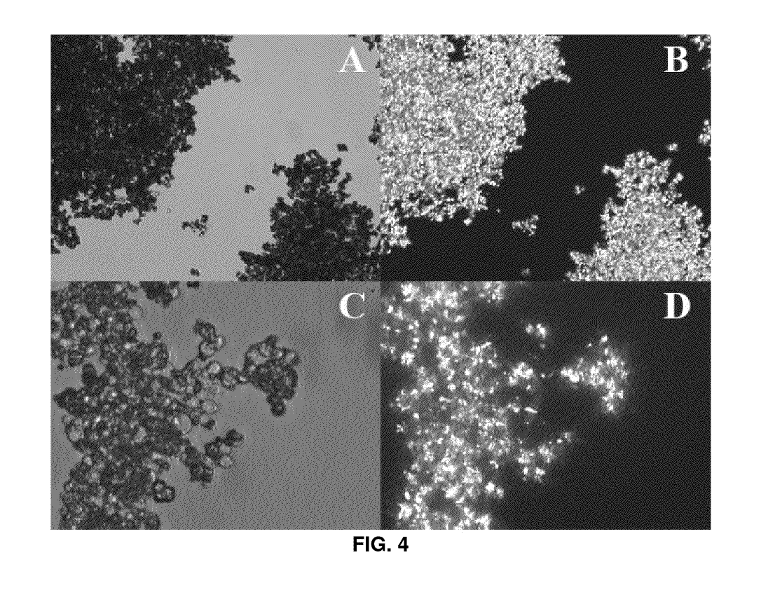

FIGS. 3A and 3B illustrate the effect of yeast cells on mineralization of calcium carbonate. FIG. 3A is a micrograph of crystals formed in the presence of S. cerevisiae cells; FIG. 3B, in the absence of cells. FIGS. 4A-4D show bright field (FIGS. 4A and 4C) and cross polarized light (CPL, FIGS. 4B and 4D) microscopy images of CaCO₃ mineralized in the presence of yeast expressing a mineralization peptide. FIGS. 4A and 4B are at 10× magnification; FIGS. 4C and 4D are at 40× magnification. Arrows point out crystals are attached to the cell surface.

Other embodiments are within the scope of the following claims.

1. A system for the mineralization of carbon dioxide, comprising:
a reactor containing an aqueous cell composition including a cell expressing a carbonic anhydrase on the cell surface;
a carbon dioxide source configured to supply carbon dioxide to the reactor; and
an aqueous metal ion composition including divalent metal cations, wherein the aqueous cell composition and the aqueous metal ion composition are optionally part of the same aqueous composition.

2. The system of claim 1, wherein the cell expressing the carbonic anhydrase is a yeast cell.

3. The system of claim 2, wherein the aqueous metal ion composition further includes a mineralization peptide.

4. The system of claim 3, wherein the mineralization peptide is expressed on a cell surface.

5. The system of claim 4, wherein the mineralization peptide is expressed on the surface of the cell expressing the carbonic anhydrase.

6. The system of claim 1, wherein the aqueous cell composition and the aqueous metal ion composition are not part of the same aqueous composition, and the system further comprises a separator configured to separate the cell from a solute from the aqueous cell composition; and a second reactor containing the aqueous metal ion composition.

7. The system of claim 1, wherein the carbon dioxide source includes a flue gas.

8. A method of mineralizing carbon dioxide, comprising:
providing an aqueous cell composition including a cell expressing a carbonic anhydrase on the cell surface;
contacting the aqueous cell composition with carbon dioxide, thereby producing aqueous carbonate ions; and
contacting the aqueous carbonate ions with divalent metal cations.

9. The method of claim 8, wherein the cell expressing the carbonic anhydrase is a yeast cell.

10. The method of claim 9, further comprising contacting the aqueous carbonate ions and the divalent metal cations with a mineralization peptide.

11. The method of claim 10, wherein the mineralization peptide is expressed on a cell surface.

12. The method of claim 11, wherein the mineralization peptide is expressed on the surface of the cell expressing the carbonic anhydrase.

13. The method of claim 8, wherein contacting the aqueous cell composition with carbon dioxide includes contacting the aqueous cell composition with a flue gas.

14. The method of claim 8, further comprising separating the cell expressing a carbonic anhydrase on the cell surface from the aqueous carbonate ions prior to contacting the aqueous carbonate ions with the divalent metal cations.

15. The method of claim 14, further comprising returning the separated cell to the aqueous cell composition.
